(19) United States
(12) Patent Application Publication
(10) Pub. No.: US 2026/0191765 A1
(43) Pub. Date: Jul. 9, 2026

(54) COSMETIC COMPOSITION CONTAINING AN ALCOHOL, A PLANT OIL, A FATTY ACID MONOESTER, AN EMULSIFIER AND A PLANT INGREDIENT

(71) Applicant: WELEDA AG, Arlesheim (CH)

(72) Inventors: Katharina POST, Basel (CH); Benjamin MATZKE, Leymen (FR)

(21) Appl. No.: 19/133,326

(22) PCT Filed: Nov. 29, 2023
(86) PCT No.: PCT/EP2023/083562

§ 371 Date: May 28, 2025

(30) Foreign Application Priority Data
Nov. 29, 2022 (EP) 22210393.9

(51) Int. Cl. A61K 8/9789; A61K 8/04; A61K 8/34; A61K 8/37; A61K 8/44; A61K 8/60; A61K 8/92; A61K 8/9794; A61Q 13/00

(52) U.S. Cl. CPC A61K 8/9789; A61K 8/042; A61K 8/345; A61K 8/37; A61K 8/44; A61K 8/60; A61K 8/922; A61K 8/9794; A61Q 13/00

(57) ABSTRACT

The invention relates to a cosmetic composition which is essentially plant-based, which is virtually water-free and which, when used topically on the skin, preferably dry skin, generates a pleasant warmth in contact with water, so that the skin pores are dilated and plant ingredients can advantageously penetrate the skin. The invention further relates to the preparation of such a cosmetic composition and its use, in particular as a cleansing product or rinse-off product.

The invention relates to a cosmetic composition which is essentially plant-based, which is virtually water-free and which, when used topically on the skin, preferably dry skin, generates a pleasant warmth in contact with water, so that the skin pores are dilated and plant ingredients can advantageously penetrate the skin. The invention further relates to the preparation of such a cosmetic composition and its use, in particular as a cleansing product or rinse-off product.

Weleda AG conducts research into beneficial natural plant substances for cosmetics using modern and anthroposophical methods.

As a barrier organ of the human organism, the skin, especially the epidermis, is particularly exposed to external influences (cold, chemicals, stress, environmental influences, etc.). This barrier function is maintained by skin lipids, among other things. These epidermal lipids such as glycosphingolipids, ceramides, sterols and sterol esters, fatty acids, triglycerides, n-alkanes or various polar lipids are released during the keratinization process.

In an optimal skin condition, there is a balanced ratio of skin lipids and skin moisture. This balance determines important properties of the skin, such as penetration capacity, water-binding capacity, elasticity, regenerative capacity or resistance to environmental influences and noxae of various kinds. Active plant substances, especially secondary metabolites, can have a positive influence on the skin. However, gentle products are required to care for and cleanse the skin.

The present invention is therefore based on the task of providing a cosmetic composition for topical use so that valuable plant ingredients can advantageously penetrate the skin and an additional care effect is achieved.

Surprisingly, the inventors found that the use of a cosmetic composition can be provided on an essentially vegetable basis of an oil gel, which generates a pleasant heat on contact with water, so that the skin pores can be dilated and in this way plant ingredients can advantageously penetrate the skin.

The problem is solved by the technical teaching of at least one patent claim. In particular, the invention therefore relates to an oil gel, namely a cosmetic composition comprising
a.) at least one dihydric, trihydric or tetrahydric alcohol,
b.) at least one vegetable oil,
c.) at least one fatty acid monoester,
wherein the ratio of a.):b.):c.) in % by weight is 2-4/4-6/0.5-1.5,
d.) at least one emulsifier,
e.) at least one or more plant ingredients, whereby at least one secondary plant substance is present,
f.) a maximum of 3% water by weight.
a.) to f.) is an enumeration, whereby all components of the composition a.) to f.) are different from each other or cannot be the same.

Weight specifications refer to 100% of a cosmetic composition according to the invention. Such a 100% embodiment can be taken from Example 1.

In a further embodiment of the invention, it is preferred that the ratio of a.):b.):c.) in % by weight is 3/5/1. The aforementioned ratio according to the invention is essential for the suitable viscosity for obtaining a homogeneous oil gel. Furthermore, the viscosity can be optimized by using an emulsifier d.). However, if the oil content is increased too much, too high a strength is achieved, which has a detrimental effect on homogeneous flow behavior.

In a further embodiment of the invention, it is preferred that the vegetable oil according to b.) is present in the cosmetic composition in an amount of at least 40% by weight, in particular 50% by weight.

Furthermore, the water content should not be more than 3% by weight, preferably 2% by weight. Otherwise there is no clear, flowable oil gel, but clouding occurs, so that no advantageous homogeneity is achieved. However, due to the low water content, a special oil gel texture should be achieved which can be applied to the skin in a finely spreadable manner so that a maximum feeling of warmth or maximum warmth is created on contact with water.

On the one hand, the emulsifier according to the invention has an effect on the viscosity of the oil gel and, on the other hand, an advantageous homogeneous emulsion is formed on contact with water. Such emulsifiers can advantageously be selected from the group of non-ionic or amphoteric emulsifiers, such as sugar esters, in particular preferably sucrose esters and betaines.

In a further embodiment of the invention, it is preferred that the emulsifier according to d.) is present in the cosmetic composition at 1-6% by weight, in particular at 2-4% by weight.

After topical application or application to the skin, an emulsion is formed from the oil gel on contact with water (e.g. washing) or moistening, with pleasant heat development (negative enthalpy), so that the skin pores can be dilated and in this way plant ingredients can penetrate the skin in a beneficial way.

This is particularly advantageous for use as a cleansing product or rinse-off product, where the product is washed off or removed using heat.

The oil gel according to the invention comprises:
a.) Divalent, trivalent or tetravalent alcohols are those selected from the group consisting of ethanediol, glycerol, propanediol, erythritol, butanediol, C1-C4 alcohols with 2, 3 or 4 OH groups. Glycerol is preferred according to the invention.
b.) For the purposes of the present invention, vegetable oil means a tri- or diglyceride in which at least one fatty acid is mono- or polyunsaturated, in particular fatty acids with 14 to 26 carbon atoms, in particular oleic acid, linoleic acid or linolenic acid. The vegetable oil is liquid at room temperature. Such native vegetable oils may contain further substituents, such as OH, NH₍₂₎, etc. Such vegetable oils are obtained by various methods, such as pressing (cold pressing, hot pressing), extraction with solvents (heptane, petroleum ether, etc.), superfluid chromatography, etc.
   One or more vegetable oils within the meaning of the present invention may be selected from the group consisting of, but not limited to, açaí oil, algae oil, argan oil, avocado oil, babaçu oil, borage oil, cupuaçu butter, safflower oil, peanut oil, hazelnut oil, hemp oil, jatropha oil, jojoba oil, cocoa butter, coconut oil, pumpkin seed oil, linseed oil, macadamia oil, corn oil, almond oil, mango butter, apricot kernel oil, evening primrose oil, olive oil, palm oil, palm kernel oil, papaya oil, pistachio oil, pecan oil, perilla oil, rapeseed oil, rice oil, castor oil, mustard oil, sesame oil, shea butter, soybean oil, sunflower oil, tung oil, walnut oil, watermelon seed oil, grape seed oil, cedar oil.
c.) A fatty acid monoester in the sense of the present invention means that a fatty acid is esterified with an alcohol, so that the fatty acid monoester preferably has 16-26 C atoms, also called ester oil, in particular isoamyl laurate is preferred. Furthermore, the fatty acid can be branched or unbranched, unsaturated or saturated, but saturated is preferred. It is further preferred that the alcohol comprises more than 1C atom, is branched or unbranched, such as iso-propanol, iso-butanol, iso-pentanol and possibly has more than one OH group, such as 1,2-pentanediol. Such fatty acid monoesters can be produced from vegetable oils.
d.) Emulsifier can be selected from the group of non-ionic or amphoteric emulsifiers, such as sugar esters, in particular preferably sucrose esters and betaines.
e.) Plant ingredients according to the invention are those that contain or consist of secondary plant substances. Secondary plant substances, or secondary metabolites, are derived from products of anabolic and catabolic metabolism, in particular from carbohydrates and amino acids. Secondary plant substances according to the invention comprise in particular phenolic, isoprenoid or alkaloid compounds, such as phenols, polyphenols, flavonoids, caffeic acid derivatives, xanthones, terpenes, steroids and other natural substances. Such plant ingredients can be aqueous and/or ethanolic, or oily extracts from a plant material, in particular from leaves, roots, seeds, flowers. For example, such phytochemicals may be present in essential plant oils (e.g. terpenes, etc.). Such plant materials may be selected from the group of plant genera Aloe, Althaea, Angelica, Arnica, Artemisia, Calendula, Cannabis, Capsicum, Carum, Citrus, Centella, Echinacea, Hedeara, Humulus, Iberis, Iris, Juglans, Lepidium, Matricaria, Melissa, Mentha, Basilicum (Ocimum), Passiflora, Pelargonium, Primula, Punica, Quercus, Rosmarinus, Rumex, Salix, Salvia, Sambucus, Strychnos, Thymus, Vaccinium, Valeriana, Vebena, Viola, Vitex, Vitis.

In a further embodiment of the invention, it is preferred that “one or more plant ingredient(s), wherein at least one secondary plant substance is present” according to e.) is present in the cosmetic composition at 0.5-3% by weight, in particular at 1-2% by weight. Furthermore, e.) is materially different from b.) or c.) (supra).

The cosmetic compositions according to the invention may further comprise cosmetic excipients as commonly used in such preparations, e.g. antioxidants, free radical scavengers, preservatives, bactericides, perfumes, dyes, pigments having a coloring effect, thickening agents, surface-active substances, emollients, or other common ingredients of a cosmetic formulation such as foam stabilizers, electrolytes, and others.

The following examples of embodiments and figures serve to illustrate the invention without limiting the invention to these examples.

### EXAMPLE 1

Exemplary compositions of a composition according to the invention:

a.)

| Ingredient | Function | Weight (wt. %) | |
|---|---|---|---|
| Glycerine | Trivalent alcohol | 30% | by weight |
| Aqua | | 2% | by weight |
| Sucrose stearate | Emulsifier | 2% | by weight |
| Betaine | Emulsifier | 3% | by weight |
| Sunflower oil | Vegetable oil | 50% | by weight |
| Isoamyl laurate | Fatty acid ester | 10% | by weight |
| | (ester oil) | | |
| Rosemary, camomile, | Plant material | 1% | by weight |
| calendula, viola | | | |
| Perfume | Fragrance | 1% | by weight |
| Additive(s) | | 1% | by weight |

100

b.)

| Ingredient | Function | Weight (wt. %) | |
|---|---|---|---|
| Glycerine | Trivalent alcohol | 30% | by weight |
| Aqua | | 2% | by weight |
| Sucrose stearate | Emulsifier | 2% | by weight |
| Betaine | Emulsifier | 3% | by weight |
| Sesame oil | Vegetable oil | 51% | by weight |
| Isoamyl laurate | Fatty acid ester | 10% | by weight |
| | (ester oil) | | |
| Extracts of rosemary, | Plant material | 1% | by weight |
| camomile, calendula, | | | |
| viola | | | |
| Perfume | | 1% | by weight |

100

c.)

| Ingredient | Function | Weight (wt. %) | |
|---|---|---|---|
| Glycerine | Trivalent alcohol | 30% | by weight |
| Aqua | | 1.5% | by weight |
| Sucrose stearate | Emulsifier | 2% | by weight |
| Betaine | Emulsifier | 3% | by weight |
| Sunflower oil | Vegetable oil | 51% | by weight |
| Isoamyl laurate | Fatty acid ester | 10% | by weight |
| | (ester oil) | | |
| Extracts of rosemary, | Plant material | 1% | by weight |
| camomile, calendula, | | | |
| viola | | | |
| Perfume | | 1% | by weight |
| Additive(s) | | 0.5% | by weight |

100

d.)

| Ingredient | Function | Weight (wt. %) | |
|---|---|---|---|
| Glycerine | Trivalent alcohol | 30% | by weight |
| Aqua | | 1.5% | by weight |
| Sucrose stearate | Emulsifier | 2% | by weight |
| Betaine | Emulsifier | 3% | by weight |
| Sunflower oil | Vegetable oil | 36% | by weight |
| Coconut oil | Vegetable oil | 10% | by weight |
| Isoamyl laurate | Fatty acid ester | 15% | by weight |
| | (ester oil) | | |
| Extracts of rosemary, | Plant material | 1% | by weight |
| camomile, calendula, viola | | | |
| Perfume | | 1% | by weight |
| Additive(s) | | 0.5% | by weight |

100

e.)

| Ingredient | Function | Weight (wt. %) | |
|---|---|---|---|
| Glycerine | Trivalent alcohol | 30% | by weight |
| Aqua | | 1.5% | by weight |
| Sucrose stearate | Emulsifier | 2% | by weight |
| Betaine | Emulsifier | 3% | by weight |
| Sunflower oil | Vegetable oil | 46% | by weight |
| Shea butter | Vegetable fat | 5% | by weight |
| Castor oil | Vegetable oil | 5% | by weight |
| Isoamyl laurate | Fatty acid ester | 5% | by weight |
| | (ester oil) | | |
| Extracts of | Plant material | 1% | by weight |
| rosemary, camomile, | | | |
| calendula, viola | | | |
| Perfume | | 1% | by weight |
| Additive(s) | | 0.5% | by weight |

100

f.)

| Ingredient | Function | Weight (wt. %) | |
|---|---|---|---|
| Glycerine | Trivalent alcohol | 30% | by weight |
| Aqua | | 2% | by weight |
| Sucrose stearate | Emulsifier | 2% | by weight |
| Betaine | Emulsifier | 3% | by weight |
| Sunflower oil | Vegetable oil | 50% | by weight |
| Glyceryl caprylate | Fatty acid ester | 10% | by weight |
| | (ester oil) | | |
| Extracts of rosemary, | Plant material | 1% | by weight |
| camomile, calendula, viola | | | |
| Perfume | | 1% | by weight |
| Additive(s) | | 1% | by weight |

100

### EXAMPLE 2

### Preparation of a Composition According to the Invention:

Phase 1: Mix glycerine, water-soluble or glycerine-soluble extract (plant ingredients) and water well, then premix solids such as the emulsifier and any additives and add to phase 1, homogenize and stir until a homogeneous mixture is obtained.

Phase 2: Mix vegetable oils, ester oils and oil-soluble extracts (plant ingredients) homogeneously and heat to 70-75° C. Finally, slowly add phase 1 to phase 2, homogenize and cool to 35° C., then add perfume and any additives if necessary, homogenize again and cool to <30° C.

Depending on their solubility, the plant ingredients are added in phase 1 or phase 2.

1.-12. (canceled)

13. Oil gel containing a cosmetic composition comprising
a) at least one dihydric, trihydric or tetrahydric alcohol,
b) at least one vegetable oil,
c) at least one fatty acid monoester,
wherein the ratio of a.):b.):c.) in % by weight is 2-4/4-6/0.5-1.5,
d) at least one emulsifier,
e) at least one or more plant ingredients, whereby at least one secondary plant substance is present,
f) a maximum of 3% water by weight,
wherein all components of the composition a.) to f.) are different from each other.

14. An oil gel comprising a cosmetic composition according to claim 13, wherein the alcohol according to a.) is selected from the group consisting of ethanediol, glycerol, propanediol, erythritol, butanediol, and C1-C4 alcohols having 2, 3 or 4 OH groups.

15. Oil gel containing a cosmetic composition according to claim 13, wherein the vegetable oil according to b.) is selected from the group consisting of açaí oil, algae oil, argan oil, avocado oil, babaçu oil, borage oil, cupuaçu butter, safflower oil, peanut oil, hazelnut oil, hemp oil, jatropha oil, jojoba oil, cocoa butter, coconut oil, pumpkin seed oil, linseed oil, macadamia oil, corn oil, almond oil, mango butter, apricot kernel oil, evening primrose oil, olive oil, palm oil, palm kernel oil, papaya oil, pistachio oil, pecan nut oil, perilla oil, rapeseed oil, rice oil, castor oil, mustard oil, sesame oil, shea butter, soybean oil, sunflower oil, tung oil, walnut oil, watermelon seed oil, grape seed oil, and cedar oil.

16. Oil gel containing a cosmetic composition according to claim 13, wherein the fatty acid monoester according to c.) has 16-26 C atoms, is branched or unbranched, is unsaturated or saturated.

17. An oil gel comprising a cosmetic composition according to claim 13, wherein the ratio of a.):b.):c.) in % by weight is 3/5/1.

18. An oil gel comprising a cosmetic composition according to claim 13, wherein the vegetable oil according to b.) is present in an amount of at least 40% by weight.

19. Oil gel comprising a cosmetic composition according to claim 13 comprising at least one or more plant ingredients, wherein at least one secondary plant substance is present, selected from the group phenolic, isoprenoid or alkaloid compounds, phenols, polyphenols, flavonoids, caffeic acid derivatives, xanthones, terpenes, steroids or from aqueous and/or ethanolic or oily extracts/extracts from a plant material.

20. An oil gel comprising a cosmetic composition according to claim 19, wherein the plant material is selected from the group consisting of Aloe, Althaea, Angelica, Arnica, Artemisia, Calendula, Cannabis, Capsicum, Carum, Citrus, Centella, Echinacea, Hedera, Humulus, Iberis, Iris, Juglans, Lepidium, Matricaria, Melissa, Mentha, Basilicum (Ocimum), Passiflora, Pelargonium, Primula, Punica, Quercus, Rosmarinus, Rumex, Salix, Salvia, Sambucus, Strychnos, Thymus, Vaccinium, Valeriana, Vebena, Viola, Vitex, and Vitis.

21. An oil gel comprising a cosmetic composition according to claim 13, wherein the at least one emulsifier according to d.) is selected from nonionic or amphoteric emulsifiers, in particular sugar esters, in particular sucrose esters and betaines, and the emulsifier according to d.) is present in an amount of 1-6% by weight, in particular 2-4% by weight.

22. An oil gel comprising a cosmetic composition according to claim 13:

| | Ingredient | Weight (wt. %) | |
|---|---|---|---|
| 1.) | Glycerine | 30% | by weight |
| 2.) | Aqua | 2% | by weight |
| 3.) | Sucrose stearate | 2% | by weight |
| 4.) | Betaine | 3% | by weight |
| 5.) | Sunflower oil | 50% | by weight |
| 6.) | Isoamyl laurate | 10% | by weight |
| 7.) | Rosemary and/or camomile and/or | 1% | by weight |
| | calendula and/or viola extracts | | |
| 8.) | Perfume | 1% | by weight |
| 9.) | Other cosmetic additives | 1% | by weight |

23. An oil gel comprising a cosmetic composition according to claim 13, which forms an emulsion on contact with water while generating heat.

24. Rinse-off product comprising an oil gel according to claim 13.
